(19) United States
(12) Patent Application Publication
(10) Pub. No.: US 2025/0135112 A1
(43) Pub. Date: May 1, 2025

(54) FLOW RATE CONTROL DEVICE AND METHODS FOR CANCER TREATMENT SYSTEMS

(71) Applicants: Boston Scientific Scimed, Inc., Maple Grove, MN (US); Boston Scientific Medical Device Limited, Galway (IE)

(72) Inventors: David Raab, Roseville, MN (US); Scott McGhee, Ottawa (CA); Nachiket Gole, Pune (IN); Nitesh Ghananil Baviskar, Kalyan West (IN); Bruce Fullerton, Hudson, WI (US); Randall Maas, Chaska, MN (US); Anthony Gedwillo, Minneapolis, MN (US); Mihir Sukhatme, Mumbai (IN)

(21) Appl. No.: 18/924,050

(22) Filed: Oct. 23, 2024

Related U.S. Application Data
(60) Provisional application No. 63/546,443, filed on Oct. 30, 2023.

(51) Int. Cl. A61M 5/168; A61M 5/14; A61M 5/145; A61M 5/172; A61M 5/31

(52) U.S. Cl. CPC A61M 5/16886; A61M 5/1413; A61M 5/1452; A61M 5/172; A61M 2005/3125; A61M 2205/583

(57) ABSTRACT

Embodiments herein relate to flow rate control devices and methods for use with cancer treatment systems. In an embodiment, a flow rate control device for cancer therapy systems can be included having a housing configured to engage with a fluid delivery device and a visual indicator bar. The visual indicator bar can be disposed on the housing and the flow rate control device can be configured to cause the visual indicator bar to visually change to indicate a target plunger depression pace of a plunger of the fluid delivery device. Other embodiments are also included herein.

This application claims the benefit of U.S. Provisional Application No. 63/546,443, filed Oct. 30, 2023, the content of which is herein incorporated by reference in its entirety.

### FIELD

Embodiments herein relate to flow rate control devices and methods for use with cancer treatment systems.

### BACKGROUND

According to the American Cancer Society, cancer accounts for nearly 25% of the deaths that occur in the United States each year. Cancerous tumors can form if one normal cell in any part of the body mutates and then begins to grow and multiply too much and too quickly. Cancerous tumors can be a result of a genetic mutation to the cellular DNA or RNA that arises during cell division, an external stimulus such as ionizing or non-ionizing radiation, exposure to a carcinogen, or a result of a hereditary gene mutation. Regardless of the etiology, many cancerous tumors are the result of unchecked rapid cellular division. Surgery is a common first-line therapy for many cancerous tumors. However, not every tumor can be surgically removed.

### SUMMARY

Embodiments herein relate to flow rate control devices and methods for use with cancer treatment systems. In a first aspect, a flow rate control device for cancer therapy systems can be included having a housing configured to engage with a fluid delivery device and a visual indicator bar. The visual indicator bar can be disposed on the housing and the flow rate control device can be configured to cause the visual indicator bar to visually change to indicate a target plunger depression pace of a plunger of the fluid delivery device.

In a second aspect, in addition to one or more of the preceding or following aspects, or in the alternative to some aspects, a flow rate control device can further include a control circuit. The control circuit can be configured to calculate a target plunger depression pace and cause the visual indicator bar to visually change in time sequence to indicate the same.

In a third aspect, in addition to one or more of the preceding or following aspects, or in the alternative to some aspects, visual changes of the visual indicator bar can include visual changes with a spatial dimension equal to spatial movement of the plunger consistent with the target plunger depression pace.

In a fourth aspect, in addition to one or more of the preceding or following aspects, or in the alternative to some aspects, the control circuit can be configured to receive user input regarding a target fluid flow rate and utilize the same when calculating a target plunger depression pace.

In a fifth aspect, in addition to one or more of the preceding or following aspects, or in the alternative to some aspects, the visual indicator bar can be oriented with a long axis parallel to a lengthwise axis of the plunger of the fluid delivery device.

In a sixth aspect, in addition to one or more of the preceding or following aspects, or in the alternative to some aspects, the visual indicator bar can include at least one selected from the group consisting of a visual display screen and a plurality of light emitting elements.

In a seventh aspect, in addition to one or more of the preceding or following aspects, or in the alternative to some aspects, the light emitting elements can include light emitting diodes.

In an eighth aspect, in addition to one or more of the preceding or following aspects, or in the alternative to some aspects, the flow rate control device can further include a secondary display screen, wherein the secondary display screen can be configured to display information related to fluid delivery.

In a ninth aspect, in addition to one or more of the preceding or following aspects, or in the alternative to some aspects, the information related to fluid delivery can include a target fluid flow rate.

In a tenth aspect, in addition to one or more of the preceding or following aspects, or in the alternative to some aspects, the fluid delivery device can include a syringe.

In an eleventh aspect, in addition to one or more of the preceding or following aspects, or in the alternative to some aspects, the housing or a component connected thereto can include a receiving channel or slot.

In a twelfth aspect, in addition to one or more of the preceding or following aspects, or in the alternative to some aspects, the receiving channel or slot can be configured to at least partially engage with the fluid delivery device.

In a thirteenth aspect, in addition to one or more of the preceding or following aspects, or in the alternative to some aspects, the housing can be configured to retain a barrel of a fluid delivery device via a snap-fit or pressure-fit mechanism.

In a fourteenth aspect, in addition to one or more of the preceding or following aspects, or in the alternative to some aspects, the housing or a component connected thereto can define a flange receiving slot, wherein the flange receiving slot can be configured to receive a flange of the fluid delivery device.

In a fifteenth aspect, in addition to one or more of the preceding or following aspects, or in the alternative to some aspects, the flow rate control device for cancer therapy systems can further include a finger rest, wherein the finger rest can be attached to the housing adjacent to a distal end thereof. The flow rate control device can also include finger grips, wherein the finger grips can be attached to the housing adjacent to a proximal end thereof.

In a sixteenth aspect, in addition to one or more of the preceding or following aspects, or in the alternative to some aspects, the flow rate control device can further include one or more user input elements, wherein the one or more user input elements can be disposed on the housing.

In a seventeenth aspect, a method of assisting a device user in modulating a fluid flow rate of a fluid delivery device of a cancer therapy system can be included. The method can include engaging the fluid delivery device with flow rate control device, determining a target plunger depression pace of a plunger of the fluid delivery device, and causing a visual indicator bar of the flow rate control device to visually change to indicate the target plunger depression pace.

In an eighteenth aspect, in addition to one or more of the preceding or following aspects, or in the alternative to some aspects, the method can further include causing the visual indicator bar to visually change in time sequence to indicate the target plunger depression pace.

In a nineteenth aspect, in addition to one or more of the preceding or following aspects, or in the alternative to some aspects, the method can further include receiving user input regarding a target fluid flow rate and utilizing the same when determining a target plunger depression pace.

In a twentieth aspect, in addition to one or more of the preceding or following aspects, or in the alternative to some aspects, the method can further include displaying information related to fluid delivery with the flow rate control device.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a diagram of a cancer treatment system in accordance with various embodiments herein.

FIG. 2 is a schematic view of a flow rate control device in accordance with various embodiments herein.

FIG. 3 is a schematic view of a flow rate control device in accordance with various embodiments herein.

FIG. 4 is a schematic view of a flow rate control device in accordance with various embodiments herein.

FIG. 5 is a schematic view of a flow rate control device in accordance with various embodiments herein.

FIG. 6 is a flow chart of operations of a method in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

As referenced above, surgery is a common first-line therapy for many cancerous tumors. However, not every tumor can be surgically removed. Another approach to treating some types of cancer can include delivering radiation to cancerous tissue to destroy cancerous cells therein. In one approach, microspheres such as small glass microspheres that have Y-90 mixed into the glass can be deposited in or near a cancerous tumor and beta radiation emitting therefrom can damage DNA of the cancerous cells inducing apoptosis and cell cycle arrest.

Delivery of microspheres can be performed using a catheter, such as a microcatheter placed in vasculature leading to a tumor, such as an artery supplying blood to the tumor. The microspheres can be combined with a fluid, such as saline, to form a suspension that is then directed through the catheter to the site of deposition.

In some approaches, a clinician must utilize a syringe or similar device to deliver the fluid to be mixed with the microspheres. The clinician advances the plunger of the syringe at a desired pressure/speed resulting in a particular flow rate of fluid through the system for a particular amount of time to a achieve a target volume of suspension delivered out of the catheter and into the patient. However, it is tedious and potentially confusing for system users to manage aspects of this process. For example, it is difficult for system users to depress the plunger at the correct pace in order to achieve a target flow rate (such as between 5 ml/min and 20 ml/min, or within other flow rate bounds). Possible consequences of depressing the plunger at an incorrect pace could include incomplete delivery of microspheres to the patient (too low flow rate) or microspheres flowing to the wrong location in the vasculature (too high flow rate).

Systems and methods herein can be used to assist a device user in using a fluid delivery device to provide a target fluid flow rate. For example, a visual indicator bar can be used to provide a visual cue to the device user so they know how fast they should be depressing a plunger (or similar element) of a fluid delivery device to achieve a target fluid flow rate. In various embodiments, flow rate control devices for cancer therapy systems herein can include a housing that is configured to engage with a fluid delivery device (such as a syringe or other fluid delivery device). A visual indicator bar can be disposed on the housing and the flow rate control device can be configured to cause the visual indicator bar to visually change (such as sequentially turning on or off visually distinct spots arranged in a line along the visual indicator bar to cause the visual effect of a moving target or moving line to indicate a target plunger depression pace of a plunger of the fluid delivery device. In some embodiments, turning on or off light emitting elements such as LEDs arranged in a line can cause the sensation of a moving target or moving line. In some embodiments, the device can display a moving progress bar or line within the visual indicator bar, or the like). In this manner, the device user only needs to match the movement speed illustrated by the flow rate control device to achieve a target plunger depression rate and therefore a target flow rate.

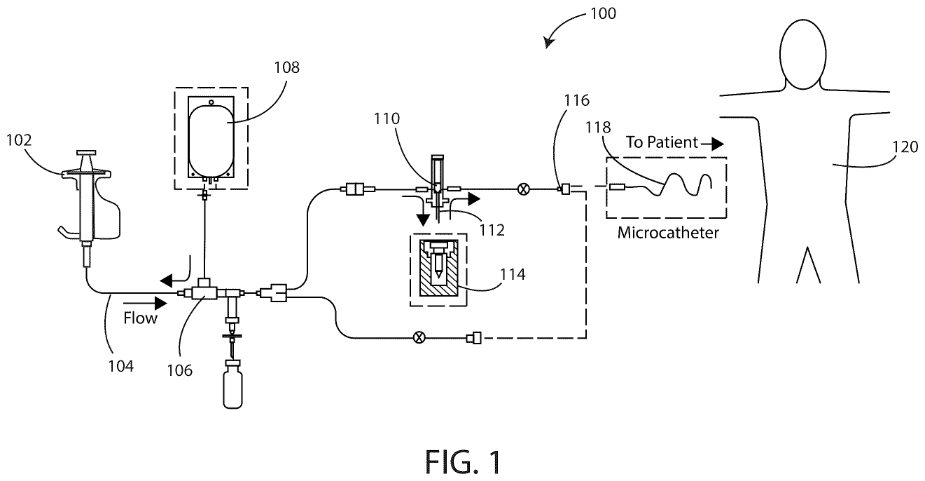

Referring now to FIG. 1, a diagram of a cancer treatment system 100 is shown in accordance with various embodiments herein. Major parts of the cancer treatment system 100 include a therapeutic fluid delivery device assembly 102 including a fluid delivery device engaged with a flow rate control device described in more detail below. The cancer treatment system 100 can also include a fluid supply tube 104 and a flow control valve 106. The cancer treatment system 100 also includes a saline supply reservoir 108. The cancer treatment system 100 also includes a fluid injection and suspension withdrawal device 110. The cancer treatment system 100 also includes a radioactive microsphere supply reservoir 114. The cancer treatment system 100 also includes an outflow port 116. The cancer treatment system 100 also includes a microcatheter 118. FIG. 1 also shows a patient 120 in which the microcatheter 118 can be inserted to deliver the therapeutic suspension of microspheres.

In use, (omitting some possible operations for ease of explanation) the clinician or other system user can pull back on the plunger (or similar element) of a fluid delivery device causing fluid (such as saline) to be withdrawn from the saline supply reservoir 108, through the flow control valve 106 and the fluid supply tube 104, and into the fluid delivery device. Alternatively, the fluid delivery device can be preloaded with fluid. The flow rate control device can then be triggered to initiate a guidance phase where a visual indicator bar visually demonstrates an appropriate depression pace/speed for the plunger. With this guidance, the clinician or other system user can depress the plunger causing fluid to flow from the therapeutic fluid delivery device, through the fluid supply tube 104, through the flow control valve 106, and into the fluid injection and suspension withdrawal device 110. The fluid injection and suspension withdrawal device 110 can be in fluid communication with the radioactive microsphere supply reservoir 114 and can direct a flow of fluid into the radioactive microsphere supply reservoir 114 coming from the therapeutic fluid delivery device 102 such as through one of a pair of needles, cannulas, or tubes 112. The fluid can become mixed with microspheres in the radioactive microsphere supply reservoir 114 forming a suspension which can then exit via the fluid injection and suspension withdrawal device 110 via another needle, cannula, or tube 112 and through tubing and out of the outflow port 116 and into the microcatheter 118 and into a desired site of the patient 120. It will be appreciated that various other operations can also be performed including, but not limited to, system priming, bubble removal, one or more flushing operations, and the like.

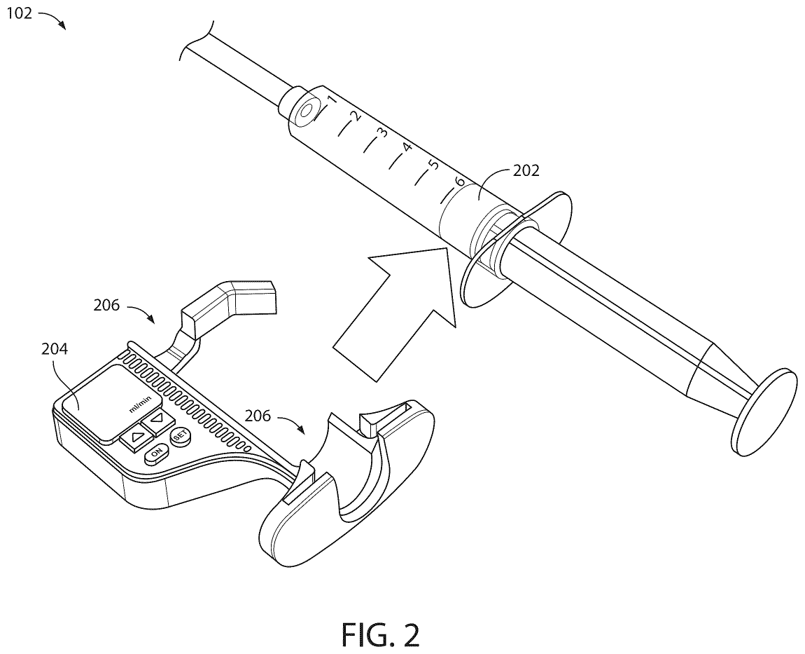

Referring now to FIG. 2, a schematic view of a flow rate control device is shown in accordance with various embodiments herein. FIG. 2 specifically shows a therapeutic fluid delivery device assembly 102 including a fluid delivery device 202 (such as a syringe) and a flow rate control device 204. As illustrated, the flow rate control device 204 can be fitted to the fluid delivery device 202. For example, a portion of the fluid delivery device 202 can fit within receiving channel or slot 206. In some cases, the flow rate control device 204 can retain a barrel of a fluid delivery device 202 via a snap-fit or pressure-fit mechanism. However, other means of keeping the flow rate control device 204 together with the fluid delivery device 202 are also contemplated herein including clips, straps, bands, snaps, adhesives, and the like. In some embodiments, the flow rate control device may not be connected to the fluid delivery device, but still positioned adjacent thereto in order to provide a visual pacing guide for proper plunger depression speed.

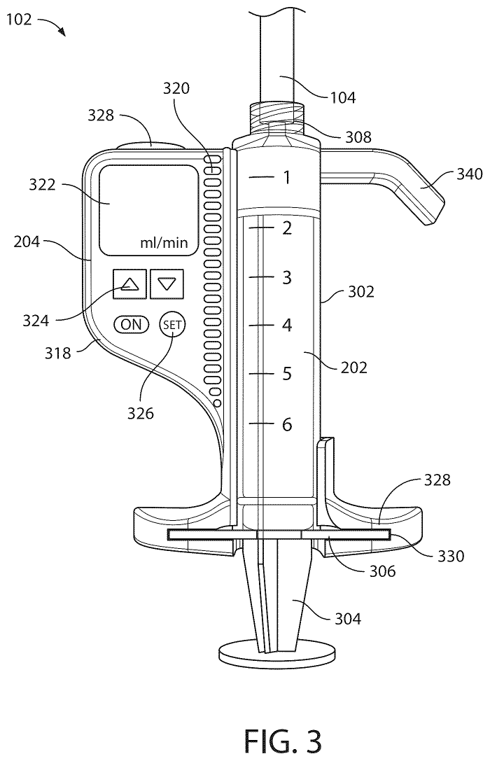

Referring now to FIG. 3, a schematic view of a flow rate control device is shown in accordance with various embodiments herein. In specific, FIG. 3 shows a therapeutic fluid delivery device assembly 102 including a fluid delivery device 202 along with a flow rate control device 204. In this view, the fluid delivery device 202 is connected to fluid supply tube 104. In this example, the fluid delivery device 202 includes a barrel 302 and a plunger 304 that is configured to slide within an interior volume of the barrel 302 to generate pressure when the plunger 304 is depressed causing fluid to flow out of the fluid delivery device 202 via a fluid orifice 308 and into the fluid supply tube 104.

In this example, the flow rate control device for cancer therapy systems 204 includes a housing 318 and a visual indicator bar 320 disposed thereon. In this particular case, the flow rate control device for cancer therapy systems 204 also includes a secondary display screen 322. In various embodiments, the secondary display screen 322 can be configured to display information related to fluid delivery, such as a target fluid delivery flow rate. In some embodiments, the secondary display screen 322 can be integrated with visual indicator bar 320, such as both being part of a single display screen (e.g., such that a visual indicator bar is displayed on a display screen with or without other visual elements). However, in other embodiments the visual indicator bar 320 and the secondary display screen 322 can be physically separate.

The flow rate control device for cancer therapy systems 204 also includes a first input element 324, a second user input element 326, and a third user input element 328, though various numbers of user input elements (real or virtual such as displayed on a screen), such as buttons, switches, toggles, are contemplated herein. The user input elements can be used to perform various functions including, but not limited to, turning on the device, starting a delivery/guidance phase, inputting information regarding a target fluid flow rate, inputting information regarding a fluid delivery device size, or any other operational or configuration information.

In this example, the flow rate control device for cancer therapy systems 204 also includes finger grips 328 and a finger rest 340 attached to the housing 318. In this case, the finger grips 328 are attached to the housing 318 adjacent to a proximal end thereof and the finger rest 340 can be attached to the housing 318 adjacent to a distal end thereof. The finger rest 340 and finger grips 328 can make it easier for a user to manipulate the device. The finger rest 340 can extend outward largely perpendicular to a lengthwise axis of the fluid delivery device 202 and, in some embodiments, include an angled terminal portion such as shown in FIG. 3.

The housing 318 of the flow rate control device 204 can be configured to engage with the fluid delivery device 202 and/or elements connected thereto. In this example, the housing 318 and/or the portion of the housing 318 forming the finger grips 328 include a flange receiving slot 330 therein. The fluid delivery device 202 can include a flange 306 that can fit within the flange receiving slot 330. The fluid delivery device 202 can be securely connected to the flow rate control device 204 when the flange 306 is inserted into the flange receiving slot 330. Alternatively or in addition, in some embodiments, the flow rate control device 204 can be secured to the fluid delivery device 202 using a snap-fit or pressure-fit mechanism. In some embodiments, various straps, clips, belts, or other mechanisms can be used to secure the flow rate control device 204 and the fluid delivery device 202 together. In some embodiments, the finger rest 340 can define a channel or U-shaped portion into which the barrel or other structure of the fluid delivery device 202 can fit and/or be secured.

The visual indicator bar 320 can include various specific components to generate a visual guide observable by the device user. In some embodiments, the visual indicator bar 320 can include at least one selected from the group consisting of a visual display screen (such as an LCD screen) and a plurality of light emitting elements (such as LEDs or other light emitting components).

In various embodiments, visual changes of the visual indicator bar 320 include visual changes with a spatial dimension/speed equal to a desired spatial movement/speed of the plunger 304 consistent with (such as matching) a target plunger depression pace. In this manner, achieving a target plunger depression pace becomes as easy and intuitive as matching the pace indicated by the visual indicator bar 320. For example, assume that a target flow rate is 12 milliliters per minute. If a 6 milliliter syringe is being used as the fluid delivery device, then a full stroke (or full depression) of the plunger needs to occur in 30 seconds. If the distance of plunger depression for a full stroke is 6 centimeters, then achieving a target flow rate requires moving the plunger at a pace of 12 centimeters per minute (as merely one hypothetical example-many different specific paces are contemplated herein). The visual indicator bar 320 can provide the visual effect of movement at the same pace of 12 centimeters per minute to make it easy for the device user to achieve the same pace with the plunder of the fluid delivery device. For example, if the visual indicator bar 320 includes a plurality of LEDs arranged in a line over a distance of 6 centimeters (e.g., matching the full stroke distance of the fluid delivery device) and spaced so that there was 1 LED (or other light emitting element) per centimeter, then achieving a speed of 12 centimeters per minute would be a matter of changing the lighting state of at least one LED every 5 seconds. It will be appreciated changing the lighting state could include turning one or more additional LEDs on (in the case of a “count up” scenario), turning one or more additional LEDs off (in the case of a “count down” scenario), or both turning one or more LEDs on and turning one or more LEDs off (in the case of a “moving” lit element, “moving” unlit element, or blinking target point). Similarly, a progress bar, progress line, (or any other visual element) can be displayed and shown to move at a speed of 12 centimeters per minute. It will be appreciated that changes of light emitting elements (LEDs including OLEDs, other types of lights, pixels, etc.) are not confined to be turned off or on to achieve visual guidance as described herein. For example, such elements could instead or in addition change color and/or change intensity to provide the visual appearance of motion.

In various embodiments, the visual indicator bar 320 can be oriented with a long axis parallel to a lengthwise axis of the plunger 304 of the fluid delivery device 202. This orientation can make it even easier for a device user to match the pace of plunger 304 depression with the target pace as indicated by the visual indicator bar 320. In some embodiments, the target point, target line of the visual indicator bar 320 used to create the appearance of motion can be configured to line up with a distal end of the plunger of a fluid delivery device. The distal end of the plunger can be visually distinct from other portions of the plunger making it easy to track plunger position against the visual indicator bar 320. For example, the distal end of the plunger can include a dosage line or other visually distinct element. In some embodiments, the distal end of the plunger can include a black elastomeric (or another material) plunger seal that is visually distinct from other portions of the plunger.

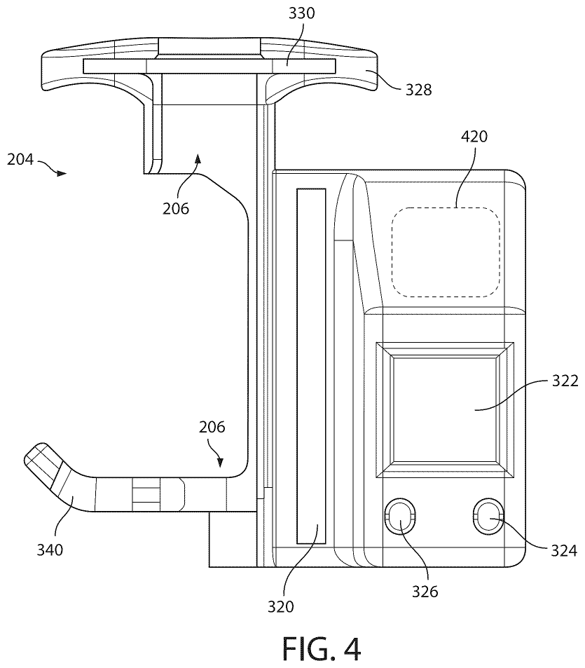

Referring now to FIG. 4, a schematic view of a flow rate control device 204 is shown in accordance with various embodiments herein. As before, the flow rate control device 204 includes a visual indicator bar 320 and a secondary display screen 322. The flow rate control device 204 also includes a first input element 324 and a second input element 326. The flow rate control device for cancer therapy systems 204 also includes finger grips 328 and a finger rest 340. The finger grips 328 portion includes a flange receiving slot 330. In various embodiments, the receiving channel or slot 206 can be configured to at least partially engage with a fluid delivery device.

The flow rate control device for cancer therapy systems 204 can also include control circuitry 420 disposed therein. The control circuitry 420 can include various components to execute operations herein including one or more of a microprocessor, an application specific integrated circuit (ASIC), memory circuit (such as random access memory (RAM), read only memory (ROM)) and/or Electrically Erasable ROM (EEROM/Flash), recorder circuitry, controller circuit, a telemetry circuit, a power supply circuit (including, for example, a battery), a timing circuit, and, a recharging circuit, amongst others components.

The control circuitry 406 can control the visual indicator bar 320 and a secondary display screen 322. The control circuitry 406 can process input as received from the first input element 324 and a second input element 326, and/or input received from a wired or wireless connection. In various embodiments, the control circuit 420 can be configured to receive user input regarding a target fluid flow rate and utilize the same when calculating a target plunger 304 depression pace. The control circuitry 406 can also perform various calculations herein. In various embodiments, the control circuit 420 can be configured to calculate a target plunger 304 depression pace and cause a visual indicator bar 320 to visually change in time sequence to indicate the same. In various embodiments, visual changes of a visual indicator bar 320 include visual changes with a spatial dimension/speed equal to spatial movement/speed of the plunger 304 consistent with a target plunger 304 depression pace. For example, if the plunger needs to move at a speed of 6 cm in 30 seconds in order to achieve a particular target fluid flow rate, then the visual indicator bar 320 can include a visual feature that appears to move or otherwise visually reflect movement at the same pace.

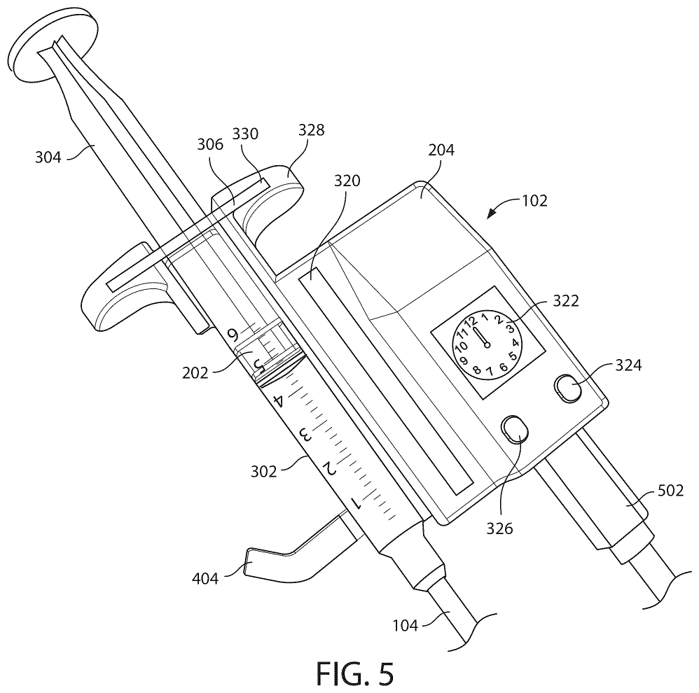

Referring now to FIG. 5, a schematic view of a flow rate control device is shown in accordance with various embodiments herein. FIG. 5 shows a therapeutic fluid delivery device assembly 102 along with a fluid supply tube 104. As before, the therapeutic fluid delivery device assembly 102 includes a fluid delivery device 202 along with a flow rate control device 204.

The fluid delivery device 202 includes a barrel 302, a plunger 304, and a flange 306. The flow rate control device 204 includes a visual indicator bar 320. The flow rate control device 204 also includes a secondary display screen 322. The flow rate control device 204 also includes a first user input element 324 and a second user input element 326. The flow rate control device for cancer therapy systems 204 also includes finger grips 328 defining a flange receiving slot 330, and a finger rest 340. In this example, the flow rate control device 204 also includes a power supply/data transmission cord 502. The cord 502 can be used to provide power to the flow rate control device 204 and/or provide for and exchange of signals and/or data with a separate system or device.

### Methods

Many different methods are contemplated herein, including, but not limited to, methods of making, methods of using, methods of controlling a fluid flow rate, and the like. Aspects of system/device operation described elsewhere herein can be performed as operations of one or more methods in accordance with various embodiments herein.

In various embodiments, operations described herein and method steps can be performed as part of a computer-implemented method executed by one or more processors of one or more computing devices. In various embodiments, operations described herein and method steps can be implemented instructions stored on a non-transitory, computer-readable medium that, when executed by one or more processors, cause a system to execute the operations and/or steps.

In an embodiment, a method of assisting a device user in modulating a fluid flow rate of a fluid delivery device of a cancer therapy system is included. The method can include engaging the fluid delivery device with flow rate control device, determining a target plunger depression pace of a plunger of the fluid delivery device, and causing a visual indicator bar of the flow rate control device to visually change to indicate the target plunger depression pace. In an embodiment, the method can further include causing the visual indicator bar to visually change in time sequence to indicate the target plunger depression pace. In an embodiment, the method can further include receiving user input regarding a target fluid flow rate and utilizing the same when determining a target plunger depression pace. In an embodiment, the method can further include displaying information related to fluid delivery with the flow rate control device.

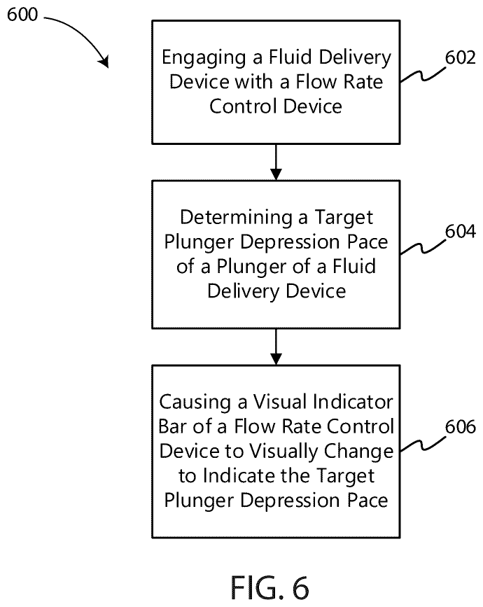

Referring now to FIG. 6, a flow chart of operations of a method is shown in accordance with various embodiments herein. FIG. 6 shows a method of assisting a device user in modulating a fluid flow rate of a fluid delivery device of a cancer therapy system 600. The method can include an engaging a fluid delivery device with flow rate control device 602. The method can also include determining a target plunger depression pace of a plunger of a fluid delivery device 604. The method can also include causing a visual indicator bar of flow rate control device to visually change to indicate the target plunger depression pace 606.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A flow rate control device for cancer therapy systems comprising:
a housing, wherein the housing is configured to engage with a fluid delivery device; and
a visual indicator bar, wherein the visual indicator bar is disposed on the housing; and
wherein the flow rate control device for cancer therapy systems is configured to cause the visual indicator bar to visually change to indicate a target plunger depression pace of a plunger of the fluid delivery device.

2. The flow rate control device for cancer therapy systems of claim 1, further comprising a control circuit, wherein the control circuit is configured to calculate a target plunger depression pace and cause the visual indicator bar to visually change in time sequence to indicate the same.

3. The flow rate control device for cancer therapy systems of claim 2, wherein visual changes of the visual indicator bar include visual changes with a spatial dimension equal to spatial movement of the plunger consistent with the target plunger depression pace.

4. The flow rate control device for cancer therapy systems of claim 2, wherein the control circuit is configured to receive user input regarding a target fluid flow rate and utilize the same when calculating a target plunger depression pace.

5. The flow rate control device for cancer therapy systems of claim 1, wherein the visual indicator bar is oriented with a long axis parallel to a lengthwise axis of the plunger of the fluid delivery device.

6. The flow rate control device for cancer therapy systems of claim 1, the visual indicator bar comprising at least one selected from the group consisting of a visual display screen and a plurality of light emitting elements.

7. The flow rate control device for cancer therapy systems of claim 6, the light emitting elements comprising light emitting diodes.

8. The flow rate control device for cancer therapy systems of claim 1, further comprising a secondary display screen, wherein the secondary display screen is configured to display information related to fluid delivery.

9. The flow rate control device for cancer therapy systems of claim 8, the information related to fluid delivery comprising a target fluid flow rate.

10. The flow rate control device for cancer therapy systems of claim 1, the fluid delivery device comprising a syringe.

11. The flow rate control device for cancer therapy systems of claim 1, the housing comprising a receiving channel or slot.

12. The flow rate control device for cancer therapy systems of claim 11, wherein the receiving channel or slot is configured to at least partially engaged with the fluid delivery device.

13. The flow rate control device for cancer therapy systems of claim 1, wherein the housing is configured to retain a barrel of a fluid delivery device via a snap-fit or pressure-fit mechanism.

14. The flow rate control device for cancer therapy systems of claim 1, the housing defining a flange receiving slot, wherein the flange receiving slot is configured to receive a flange of the fluid delivery device.

15. The flow rate control device for cancer therapy systems of claim 1, further comprising:
a finger rest, wherein the finger rest is attached to the housing adjacent to a distal end thereof; and
finger grips, wherein the finger grips are attached to the housing adjacent to a proximal end thereof.

16. The flow rate control device for cancer therapy systems of claim 1, further comprising one or more user input elements, wherein the one or more user input elements are disposed on the housing.

17. A method of assisting a device user in modulating a fluid flow rate of a fluid delivery device of a cancer therapy system comprising:
engaging the fluid delivery device with flow rate control device;
determining a target plunger depression pace of a plunger of the fluid delivery device; and
causing a visual indicator bar of the flow rate control device to visually change to indicate the target plunger depression pace.

18. The method of claim 17, further comprising causing the visual indicator bar to visually change in time sequence to indicate the target plunger depression pace.

19. The method of claim 17, further comprising receiving user input regarding a target fluid flow rate and utilizing the same when determining the target plunger depression pace.

20. The method of claim 17, further comprising displaying information related to fluid delivery with the flow rate control device.
